(19) United States
(12) Patent Application Publication
(10) Pub. No.: US 2026/0273251 A1
(43) Pub. Date: Sep. 17, 2026

(54) FLAVORED TOPICAL ANESTHETIC FORMULA AND APPLICATOR

(71) Applicant: Tamara Lee Jimenez, Mississauga (CA)

(72) Inventor: Tamara Lee Jimenez, Mississauga (CA)

(21) Appl. No.: 19/028,610

(22) Filed: Jan. 17, 2025

(51) Int. Cl. A61M 35/00

(52) U.S. Cl. CPC A61M 35/003; A61M 2202/048

(57) ABSTRACT

A topical anesthetic formulation and dosage container that is utilized to store and apply a topical analgesic wherein the formulation includes a percentage of a flavoring agent. The container includes a single dose vial body having an interior volume configured to receive and store the formulation. The formulation has a viscous gel-like consistency and includes at least five percent composition of a flavoring agent and further includes a composition range of analgesics from five to thirty percent. The vial body has an integrally formed applicator member that is configured to be fragmented from the upper end of the vial body for use and application of the formulation. The applicator member includes an upper portion and lower portion wherein the lower portion has a brush tip.

### FIELD OF THE INVENTION

The present invention relates generally to topical anesthetics, more specifically but not by way of limitation, a topical anesthetic for use in providing numbing of lips prior to a lip procedure wherein the present invention includes in the formulation thereof a flavoring agent and wherein the present invention includes a single use dosage container and applicator.

### BACKGROUND

Lip augmentation is a widely sought-after cosmetic procedure aimed at enhancing the fullness and shape of the lips. With its growing popularity, particularly in the beauty and aesthetic industries, ensuring patient comfort and minimizing procedural pain have become paramount. Numbing the lips prior to lip augmentation injections plays a critical role in achieving these goals, contributing not only to patient satisfaction but also to the overall success of the procedure. The application of a numbing agent prior to lip augmentation is a standard practice designed to mitigate the discomfort associated with the procedure. Lip injections typically involve the use of dermal fillers, such as hyaluronic acid-based products, which are administered via fine needles or cannulas. While these tools are designed to minimize trauma to the skin and underlying tissues, the sensitive nature of the lips can make the process uncomfortable. By applying a numbing agent, such as a topical anesthetic or a dental block, practitioners can significantly reduce the pain experienced during the injections.

Topical anesthetics are among the most commonly used methods for numbing the lips. These agents, which often include lidocaine or benzocaine, are applied directly to the lip area and left to penetrate the skin for a prescribed period. This approach is non-invasive and easy to administer, making it a convenient option for both practitioners and patients. However, the efficacy of topical anesthetics can vary based on factors such as the thickness of the lip tissue and the duration of application. In cases where more profound numbing is required, dental blocks may be employed. This technique involves the injection of a local anesthetic, such as lidocaine, into specific nerve sites to block pain signals. Although this method is more invasive than the application of topical agents, it offers a higher level of pain relief, making it suitable for patients with lower pain thresholds or for procedures involving extensive lip augmentation. The choice between topical anesthetics and dental blocks depends on the practitioner's assessment of the patient's needs and preferences.

Beyond physical comfort, numbing the lips also has psychological benefits for patients undergoing lip augmentation. The anticipation of pain can lead to heightened anxiety, which may negatively impact the patient's overall experience. By effectively numbing the treatment area, practitioners can alleviate these concerns, fostering a sense of trust and confidence in the procedure. This, in turn, can enhance the patient's perception of the results and encourage repeat visits for maintenance or additional cosmetic treatments. Moreover, minimizing pain during lip augmentation can contribute to better procedural outcomes. Pain-induced movements or flinching during injections can disrupt the precise placement of dermal fillers, potentially leading to uneven or suboptimal results. A numbed treatment area allows for greater control and accuracy during the procedure, ensuring that the desired aesthetic outcomes are achieved. Additionally, reduced pain may lead to a smoother recovery process, as patients are less likely to experience significant swelling or bruising related to procedural discomfort.

While numbing agents are highly effective, it is essential to consider potential risks and side effects. Topical anesthetics may cause mild irritation or allergic reactions in some patients, while dental blocks carry a small risk of nerve damage or prolonged numbness. Thorough patient assessment and a clear discussion of the benefits and risks associated with numbing techniques are crucial steps in the pre-procedure consultation process. This ensures that patients are well-informed and can make decisions that align with their comfort levels and aesthetic goals. Whether through the application of topical anesthetics or the use of dental blocks, this practice underscores the importance of patient-centered care in cosmetic medicine. Patients have expressed their dissatisfaction in regards to the poor taste of conventional numbing creams which provides a negative impact on the procedure.

Accordingly, there is a need for a dosage container and applicator for a formulation of topical anesthetic wherein the present invention includes a flavoring agent as a percentage of the formulation.

### SUMMARY OF THE INVENTION

It is the object of the present invention to provide an integrated dosage container and applicator of a proprietary formulation of topical anesthetic wherein the present invention includes a vial body having an interior volume configured to receive the topical anesthetic formulation of the present invention.

Another object of the present invention is to provide a topical anesthetic formulation that is provided in a gel-like viscosity wherein the formulation of the present invention includes utilization of anesthetics such as benzocaine, lidocaine or tetracaine.

A further object of the present invention is to provide an integrated dosage container and applicator of a proprietary formulation of topical anesthetic wherein the present invention has a releasably secured applicator member operably coupled to the vial body.

Yet a further object of the present invention is to provide a topical anesthetic formulation that is provided in a gel-like viscosity wherein the formulation of the present invention includes at least five percent of a flavoring agent.

Still another object of the present invention is to provide an integrated dosage container and applicator of a proprietary formulation of topical anesthetic wherein the applicator member includes a brush tip.

An additional object of the present invention is to provide a topical anesthetic formulation that is provided in a gel-like viscosity wherein the formulation of the present invention can be impregnated into patches or similar items.

Yet a further object of the present invention is to provide an integrated dosage container and applicator of a proprietary formulation of topical anesthetic wherein the flavoring agents include aromatic oils.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a front view of the assembled vial member of the present invention; and

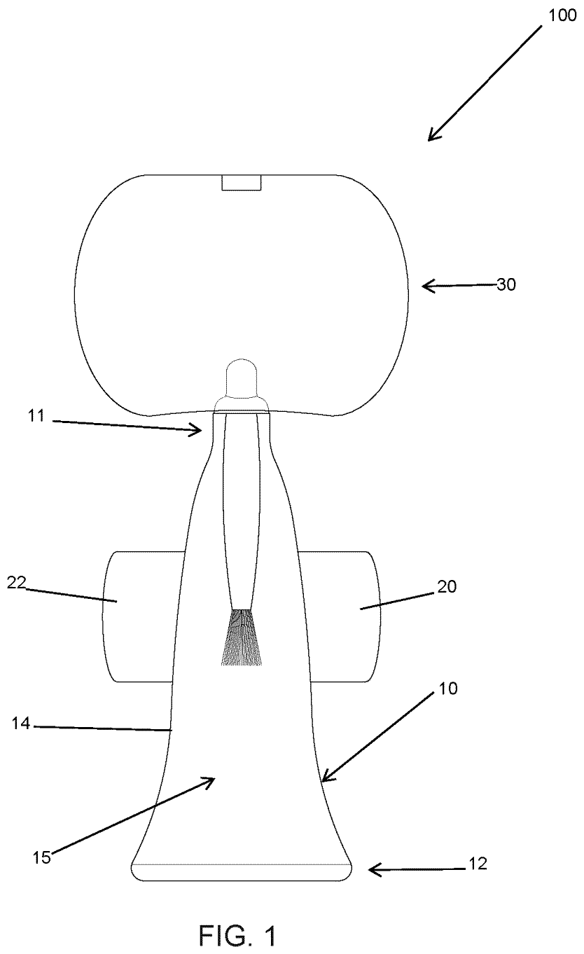

FIG. 2 is a front view of the applicator member of the present invention.

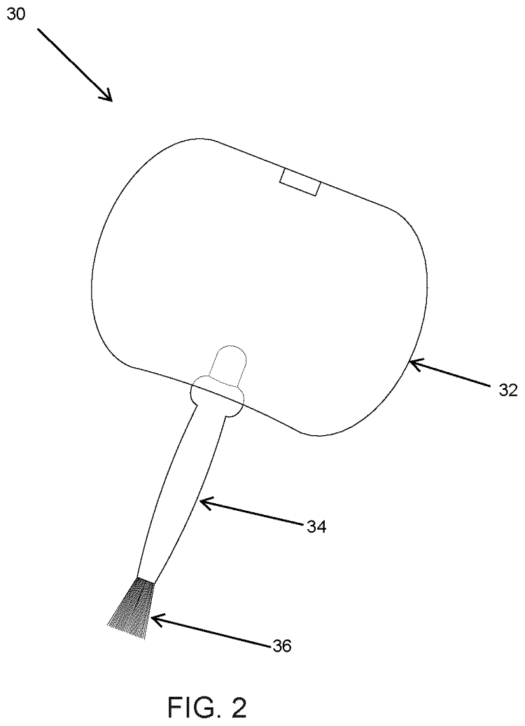

### DETAILED DESCRIPTION

Referring now to the drawings submitted herewith, wherein various elements depicted therein are not necessarily drawn to scale and wherein through the views and figures like elements are referenced with identical reference numerals, there is illustrated a topical anesthetic formulation and dosage container 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the topical anesthetic formulation and dosage container 100 includes a vial body 10 wherein the vial body 10 is manufactured from a suitable durable material such as but not limited to plastic. The vial body 10 includes upper end 11 and lower end 12 having outer wall member 14 that is formed to create an interior volume 15. The vial body 10 is tapered in form having a width at the lower end 12 that is greater than the width proximate the upper end 11. As is further discussed herein the narrower width proximate the upper end 11 assists in fragmentation of the integrally formed applicator member 30. It is contemplated within the scope of the present invention that the vial body 10 could be provided in alternate sizes and shapes wherein a desired minimum capacity for the formulation of the present invention is two milliliters. The vial body 10 includes opposing support members 20, 22 integrally formed therewith on opposing sides thereof. The support members 20,22 are planar in form extending outward perpendicularly from opposing sides of the vial body 10. The support members 20,22 provide a surface area and leverage point for a user during the process of fragmenting the applicator member 30 from the vial body 10. While the support members 20,22 have been illustrated herein having a particular shape, it is contemplated within the scope of the present invention that the support members 20,22 could be provided in alternate shapes and/or sizes in order to achieve the desired functionality discussed herein.

The applicator member 30 is integrally formed with the vial body 10. The applicator member 30 includes upper portion 32 contiguously formed with lower portion 34. Upper portion 32 is planar in manner having a sufficient surface area to provide leverage to fragment the applicator member 30 from the vial body 10. Lower portion 34 is elongated in shape being somewhat cylindrical extending downward from the upper portion 32. The length of the lower portion 34 is sufficient so as to position brush tip 36 well within the interior volume 15 of the vial body 10 to ensure contact with the formulation of the present invention disposed therein. Brush tip 36 is integrally formed on the distal end of the lower portion 34. Brush tip 36 is manufactured from a suitable material such as but not limited to nylon bristles.

The formulation of the present invention is a proprietary blend providing topical analgesic to areas such as but not limited to human lips prior to procedures performed thereon. The formulation of the present invention comprises at least fifty percent of a suitable carrier such as but not limited to coconut oil and/or shea butter. It should be understood within the scope of the present invention that the carrier could be various alternate suitable materials suitable for human skin contact. The formulation of the present invention includes at least five percent to twenty five percent of a flavoring agent. The flavoring agent provides a user of the present invention a pleasant taste to mask the other ingredients. In one embodiment of the present invention the flavoring agent is an aromatic oil having fruit and/or spice flavors. The flavoring agent composition percentage of the formulation of the present invention ranges from five to twenty percent. The formulation of the present invention comprises a combination of analgesics to provide numbing of the skin onto which the formulation is applied. The formulation includes either one or a combination of the following: lidocaine, benzocaine and/or tetracaine. An exemplary combination of the aforementioned is twenty three percent lidocaine and seven percent tetracaine. The formulation of the present invention has a percent range of the aforementioned analgesics ranging from five percent to thirty percent. It should be understood that the analgesic percentage is one of or a combination of the aforementioned analgesics.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A topical analgesic formulation and dosage container comprising:
a vial body, said vial body having a wall member forming an interior volume, said vial body having an upper end and a lower end, said vial body having an applicator member integrally formed therewith, said applicator member being formed at said upper end of said vial body, said applicator member having an upper portion and a lower portion, said lower portion of said applicator member extending into said interior volume of said vial body;
a formulation, said formulation being disposed in said interior volume of said vial body, said formulation having at least one analgesic compound, said formulation having a flavoring agent comprising a percentage thereof, said formulation having a carrier that comprises at least fifty percent of the formulation; and
wherein the applicator member is configured to be fragmented from the upper end of the vial body for application of the formulation to skin of a user.

2. The topical analgesic formulation and dosage container as recited in claim 1, wherein said formulation has a percentage of analgesic compounds ranging from five to thirty percent.

3. The topical analgesic formulation and dosage container as recited in claim 2, wherein the formulation is comprised of analgesics consisting of at least one of the following:
lidocaine, benzocaine or tetracaine.

4. The topical analgesic formulation and dosage container as recited in claim 3, wherein said flavoring agent comprises a percentage range of the formulation of five to twenty-five percent.

5. The topical analgesic formulation and dosage container as recited in claim 4, wherein said vial body further includes support members integrally formed thereon, said support members extending outward from said vial body on opposing sides thereof, said support members being planar in form.

6. The topical analgesic formulation and dosage container as recited in claim 5, wherein said lower portion of said applicator member includes a brush tip formed thereon.
